(19) United States
(12) Patent Application Publication
(10) Pub. No.: US 2009/0306672 A1
(43) Pub. Date: Dec. 10, 2009

(54) Alif inserter/distractor

(75) Inventors: Eric S. REINDEL, Encinitas, CA (US); Nathan J. MEYER, Vista, CA (US)

Correspondence Address: ALPHATEC SPINE, INC., 5818 EL CAMINO REAL, CARLSBAD, CA, 92008, US

(73) Assignee: ALPHATEC SPINE,INC., Carlsbad, CA (US)

(21) Appl. No.: 12/455,748

(22) Filed: Jun. 5, 2009

Related U.S. Application Data
(60) Provisional application No. 61/059,178, filed on Jun. 5, 2008.

(51) Int. Cl. A61B 17/60; A61B 17/58

(52) U.S. Cl. 606/90; 606/99

(57) ABSTRACT

Apparatus and method for an instrument for use in an anterior lumbar interbody fusion (ALIF) procedure for distraction of adjacent vertebrae and insertion of a vertebral body replacement (VBR) implant. The instrument includes a hollow body having a proximal end and a distal end, an inserter shaft positioned within the body configured to removably engage a VBR implant near the distal end of the body, an actuatable handle coupled to the body, the handle being configured to engage the inserter shaft to advance the VBR implant in a distal direction during handle actuation, and a pair of opposed distraction arms removably coupled to the distal end of the body, the distraction arms having paddle tips configured to fit between adjacent vertebrae, the distraction arms being movable from a closed position to an open position during distal advancement of the VBR implant between the distraction arms.

### CROSS-REFERENCES TO RELATED APPLICATIONS

The present application claims priority to U.S. Provisional Patent Application No. 61/059,178 to Reindel et al., filed Jun. 5, 2008, and entitled “ALIF INSERTER/DISTRACTOR”, the disclosure of which is incorporated herein by reference in its entirety.

### BACKGROUND OF THE INVENTION

1. Field of the Invention

The present invention relates generally to the field of spinal orthopedics, and more particularly to an inserter/disctractor for distracting adjacent vertebrae and inserting vertebral body replacements (VBR) implants within the intervertebral space during an anterior lumbar interbody fusion (ALIF) procedure.

2. Background of the Invention

The spine is a flexible column formed of a plurality of bones called vertebra. The vertebrae are hollow and piled one upon the other, forming a strong hollow column for support of the cranium and trunk. The hollow core of the spine houses and protects the nerves of the spinal cord. The different vertebrae are connected to one another by means of articular processes and intervertebral, fibrocartilaginous bodies.

The intervertebral fibro-cartilages are also known as intervertebral disks and are made of a fibrous ring filled with pulpy material. The disks function as spinal shock absorbers and also cooperate with synovial joints to facilitate movement and maintain flexibility of the spine. When one or more disks degenerate through accident or disease, nerves passing near the affected area may be compressed and are consequently irritated. The result may be chronic and/or debilitating back pain. Various methods and apparatus have been designed to relieve such back pain, including spinal fusion using an interbody spacer using techniques such as anterior lumbar interbody fusion (ALIF) surgical technique. The implants used in these techniques, also commonly referred to as vertebral body replacements (VBR) implants, are placed in the interdiscal space between adjacent vertebrae of the spine.

The VBR implant is designed to be inserted anteriorly into the spine after the intervertebral disk is removed. One problem for insertion of an implant is that the adjacent vertebra move closer together other once the natural disc tissue is removed. These vertebras must be separated to enable the placement of the VBR implant. Separating the vertebrae may be done using a distractor, and then the VBR implant may be positioned between the vertebrae using an inserter.

There exists a need for an improved implantation/distraction instruments for distracting adjacent vertebrae and inserting VBR implants within the intervertebral space during an ALIF procedure.

### BRIEF SUMMARY OF THE INVENTION

In a first aspect, embodiments of the present invention provide an instrument for use in an anterior lumbar interbody fusion (ALIF) procedure for distraction of adjacent vertebrae and insertion of a vertebral body replacement (VBR) implant. The instrument includes a hollow body having a proximal end and a distal end, an inserter shaft positioned within the body configured to removably engage a VBR implant near the distal end of the body, an actuatable handle coupled to the body, the handle being configured to engage the inserter shaft to advance the VBR implant in a distal direction during handle actuation, and a pair of opposed distraction arms removably coupled to the distal end of the body, the distraction arms having paddle tips configured to fit between adjacent vertebrae, the distraction arms being movable from a closed position to an open position during distal advancement of the VBR implant between the distraction arms.

In many embodiments, the opposed distraction arms include opposed ramps configured to contact the VBR implant during distal advancement to move the opposed distraction arms from the closed position to the open position.

In many embodiments, the paddle tips are selected to match a width of the desired VBR implant.

In many embodiments, each distraction arm includes a depressible button configured for coupling/uncoupling the distraction arm from the body.

In many embodiments, the distraction arms are made of titanium or aluminum.

In many embodiments, the inserter shaft further includes endposts proximate the VBR implant, the endposts being configured to contact the adjacent vertebrae once the VBR implant is positioned between the adjacent vertebrae

In many embodiments, the handle actuation includes depressing the handle toward the body, each handle depression advancing the VBR implant distally at least 1 cm.

In many embodiments, the instrument further includes a handle return spring coupled to the handle and body, the return spring being configure to return the handle to a start position after each actuation.

In many embodiments, the instrument further includes a knob coupled to the inserter shaft near the proximal end of the body, the knob being configured to rotate the shaft to engage or disengage the VBR implant.

In many embodiments, the inserter shaft includes threads configured for a threaded engagement with the VBR implant.

In another aspect, embodiments of the present invention provide a system for use in an anterior lumbar interbody fusion (ALIF) procedure for distraction of adjacent vertebrae and insertion of a vertebral body replacement (VBR) implant. The system includes a hollow body having a proximal end and a distal end, an inserter shaft positioned within the body, the inserter shaft having threads on a distal end, a VBR implant in threaded engagement with inserter shaft, a knob coupled to the inserter shaft near the proximal end of the body, the knob being configured to rotate the shaft to engage or disengage the VBR implant, an actuatable handle coupled to the body, the handle being configured to engage the inserter shaft to advance the VBR implant in a distal direction during handle actuation, and a pair of opposed distraction arms removably coupled to the distal end of the body. The distraction arms include paddle tips configured to fit between adjacent vertebrae, opposed ramps configured to contact the VBR implant during distal advancement to move the opposed distraction arms from a closed position to an open position, and depressible buttons configured for coupling/uncoupling the distraction arms from the body

In many embodiments, the paddle tips are selected to match a width of the VBR implant.

In many embodiments, the distraction arms are made of titanium or aluminum.

In many embodiments, the inserter shaft further includes endposts proximate the VBR implant, the endposts being configured to contact the adjacent vertebrae once the VBR implant is positioned between the adjacent vertebrae.

In many embodiments, the handle actuation includes depressing the handle toward the body, each handle depression advancing the VBR implant distally at least 1 cm.

In many embodiments, the system further including a handle return spring coupled to the handle and body, the return spring being configure to return the handle to a start position after each actuation.

In another aspect, embodiments of the present invention provide a method of distracting adjacent vertebrae and inserting a vertebral body replacement (VBR) implant between adjacent vertebrae during an anterior lumbar interbody fusion (ALIF) procedure. The method includes positioning a VBR implant in an instrument, the instrument includes a hollow body having a proximal end and a distal end, an inserter shaft positioned within the body configured to removably engage the VBR implant, an actuatable handle coupled to the body, the handle being configured to engage the inserter shaft to advance the VBR implant in a distal direction during handle actuation, a pair of opposed distraction arms removably coupled to the distal end of the body, the distraction arms having paddle tips configured to fit between adjacent vertebrae, the distraction arms being movable from a closed position to an open position during distal advancement the VBR implant between the distraction arms. The method further includes placing the paddle tips of the distraction arms between the adjacent vertebrae, distracting the adjacent vertebrae with the paddle tips as the distraction arms move from the closed position to the open position, inserting the VBR implant between the adjacent vertebrae, disengaging the VBR implant from inserter shaft, and removing the paddle tips of the inserter/disctractor instrument from the adjacent vertebrae.

In many embodiments, the opposed distraction arms include opposed ramps configured to contact the VBR implant during distal advancement, wherein moving the distraction arms from the closed position to the open position includes actuating the handle and advancing the VBR implant between the distraction arms in contact with the ramps.

In many embodiments, disengaging the VBR implant from inserter shaft includes rotating the inserter shaft with a knob to disengage a threaded engagement with the VBR implant.

In many embodiments, the method further includes selecting distraction arms having paddle tips sized to match a width of the desired VBR implant and coupling the distraction arms to the body.

### BRIEF DESCRIPTION OF THE DRAWINGS

FIG. 1 is a perspective view showing one embodiment of an instrument 100 designed for use in anterior lumbar interbody fusion (ALIF) procedures for distraction of vertebral bodies followed by the insertion of the vertebral body replacements (VBR) implants.

FIGS. 2A and 2B are perspective views showing the distal end of the instrument shown in FIG. 1 having detachable distraction arms.

FIGS. 3A-3C shows embodiments of interchangeable distraction arms with paddle tips of different sizes.

FIG. 4 is a side view showing a VBR implant loaded onto the inner shaft of the instrument.

FIGS. 5A and 5B show side views of the handle mechanism.

FIGS. 5C and 5D show sectional views of the handle mechanism and handle.

FIG. 5E shows a perspective view of the handle mechanism and handle.

FIG. 6A is a side view and FIG. 6B is a top view showing distraction arms in the open position prior to implanting the VBR implant.

FIGS. 7A-7C show lateral views of the instrument and spine during an ALIF procedure implanting a VBR implant.

### DETAILED DESCRIPTION OF THE INVENTION

One or more detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

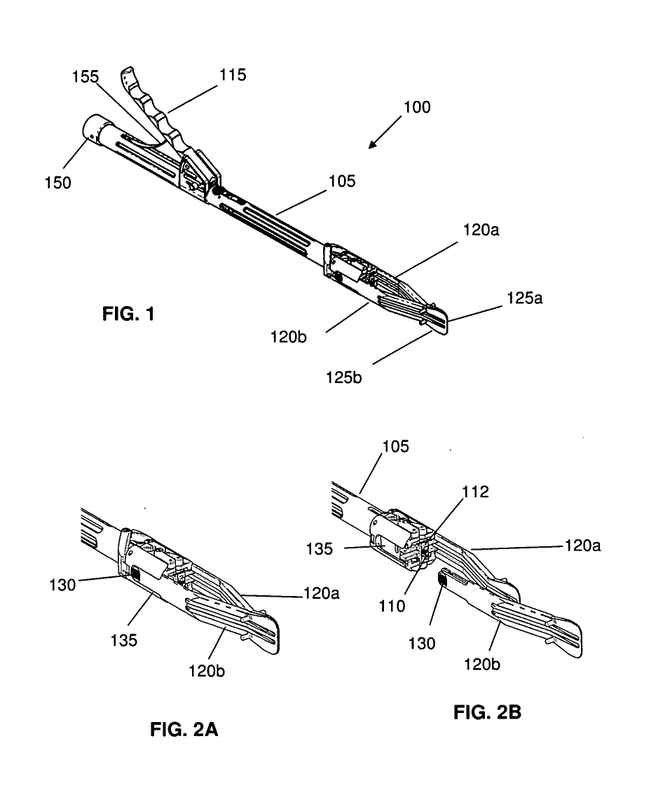

FIG. 1 is a perspective view showing one embodiment of an instrument 100 designed for use in anterior lumbar interbody fusion (ALIF) procedures for distraction of vertebral bodies followed by the insertion of the vertebral body replacements (VBR) implants. The instrument 100 includes a body 105, an inner shaft 110 extending between a proximal and distal end of the body, an actuatable handle, 115 coupled to the body, and a pair of distraction arms 120a, 120b removably coupled to the distal end of the body. The distraction arms 120a, 120b include tips 125a, 125b that are tapered to be inserted between adjacent vertebral bodies. In the embodiment shown, the paddle tips 125a, 125b are 1 mm thick at the tip and slope back at 5 degrees.

FIGS. 2A and 2B are perspective views of the distal end of the instrument 100 showing the distraction arms 120a, 120b that are detachable/interchangeable from the body. This allows the instrument 100 to use distraction arms 120a, 120b that are sized appropriately for the desired VBR implant during the procedure. The distraction arms 120a, 120b can be removed from the body 105 by depressing a latch button 130 and sliding the distraction arm out of a channel 135. Once removed, another distraction arm is inserted into the channel until the button is latched. The distraction arms may be made of metal, such as titanium or aluminum.

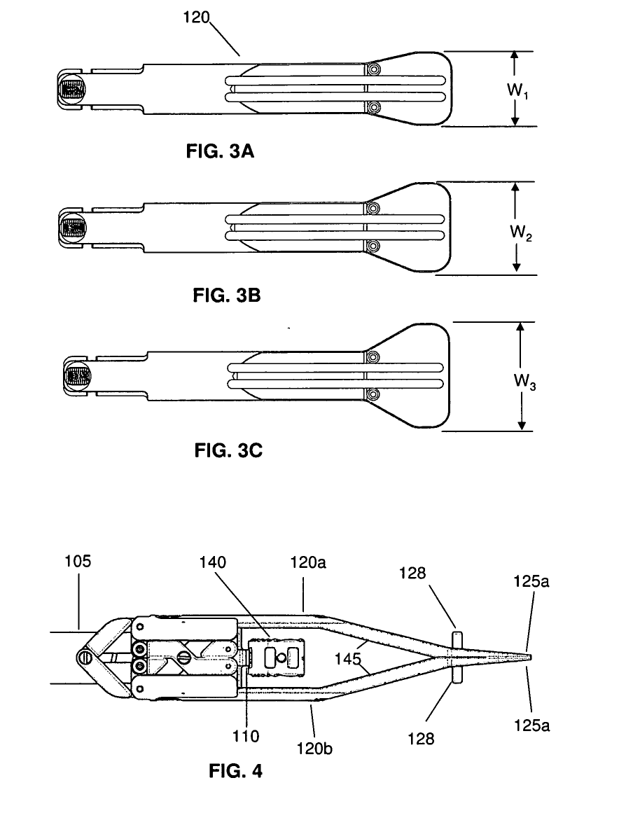

FIGS. 3A-3C shows embodiments of interchangeable distraction arms 120a, 120b with paddle tips 125 that are different is size. The tips should be wider along the perimeter than the implant, for example, 1 cm wider. In FIG. 3A the paddle tip has a width W₁ sized for a small implant, for example, an implant having a 26 mm cage. In FIG. 3B the paddle tip has a width W₂ sized for a medium implant, for example, an implant having a 32 mm cage. In FIG. 3C the paddle tip has a width W₃ sized for a large implant, for example, an implant having a 38 mm cage. Many other sizes are also contemplated.

FIG. 4 is a side view showing a VBR implant 140 loaded onto the inner shaft of the instrument 100. In one embodiment, the VBR implant is loaded onto the inner shaft by positioning two nubs 112 into two holes on the implant. See nubs 112 in FIGS. 2A and 2B. A knob 150 is coupled to the inner shaft 110 and rotated in a clockwise direction to thread on and securely hold the VBR implant 140 during insertion and distraction. Suitable implants include PEEK implants and Titanium implants sold by Alphatec Spine, Inc. As shown in the figure, the distraction arms 120a, 120b have opposing ramps 145. The distraction arms 120a, 120b further include stops 128 that contact the spine when fully inserted. The advancement of the VBR implant 140 distally forces the distraction arms 120a, 120b to spread open. The VBR implant 140 travels along 15 degree ramp angles to create the distraction prior to insertion. The resultant distraction height is dependent on the implant height. After implant insertion and end plate distraction, the VBR implant 140 is released by unscrewing the inner shaft 110 counterclockwise with the knob 150. Next the instrument 100 is removed by three (3) additional handle 115 squeeze motions. This action advances anterior stops 165 against the vertebral bodies forcing the instrument 100 to be ejected, leaving the VBR implant 140 in place. The distraction arms may include slots 166 for the anterior stops 165 to slide within.

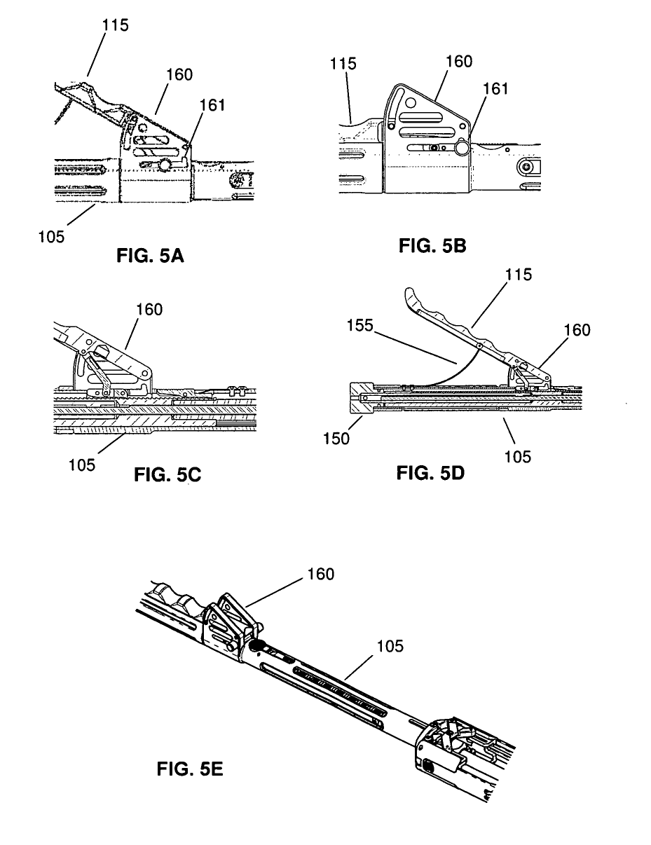

FIGS. 5A and 5B show side views of the handle mechanism 160. FIGS. 5C and 5D show sectional views of the handle mechanism 160 and handle 115. FIG. 5E shows a perspective view of the handle mechanism 160 and handle 115. The handle 115 engages the inserter shaft 110 to advance the VBR implant 140 in a distal direction during handle actuation. In the embodiment shown, the engagement may include a ratcheting mechanism where teeth within the handle mechanism 160 engage teeth on the inserter shaft 110 such that when the handle is depressed, the inserter shaft 110 moves forward advancing the VBR implant 140 distally. The handle 115 has been designed with an ergonomic grip and a handle return spring 155 to advance handle return. After the handle 115 has been depressed, the return spring 155 moves it back to the starting position. The handle mechanism 160 may also include a locking mechanism 161 to hold the handle in the down or depressed position. The handle mechanism 160 is robust and utilizes an easy release control to return to initial starting height, resetting the instrument to use again after implant release. In the embodiment shown in FIG. 5E, various levers and buttons may be used fore release control to move the system back to the starting point.

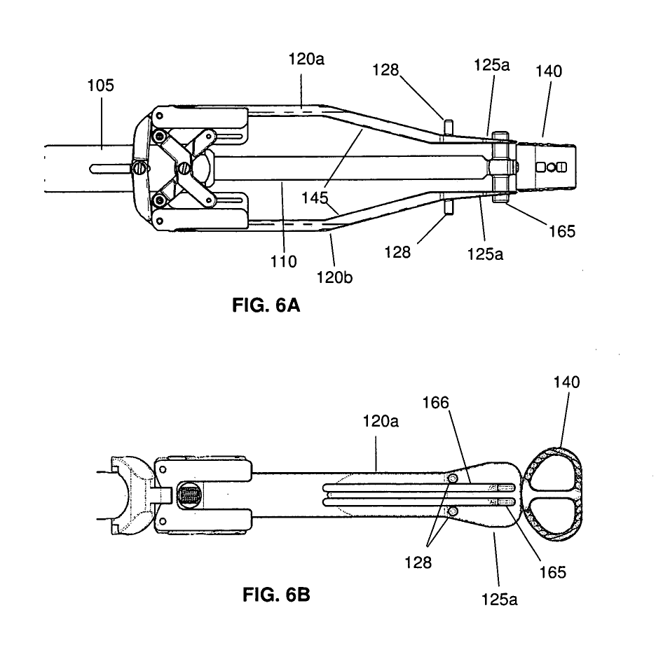

FIG. 6A is a side view and FIG. 6B is a top view showing distraction arms in the open position prior to implanting the VBR implant. The instrument 110 is simplified with a single action lever or handle 115. Each depression of the lever 115 results in 1 cm distal travel of the VBR implant 140, thus distracting the vertebrae with the distraction arms 120a, 120b as the VBR implant 140 is advanced and inserted. The advancement of the VBR implant 140 forces the distraction arms 120a, 120b to spread open. The VBR implant 140 travels along 15 degree ramp angles 140 to create the distraction prior to insertion between the vertebrae. The resultant distraction height is dependent on the implant height.

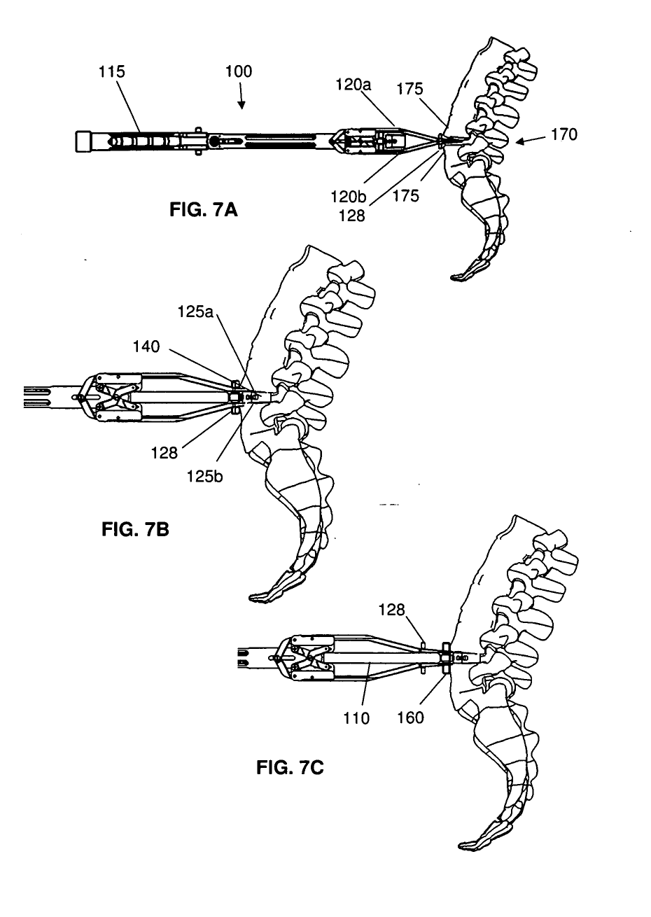

FIGS. 7A-7C show the instrument 100 in use during an ALIF procedure of the spine 170 for distraction of adjacent vertebral bodies 175 followed by the insertion of a VBR implant 140. The instrument 100 is first placed into position by placing the paddle tips 125a, 125b in the disk space between adjacent vertebrae 175 until the stops 128 contact the vertebrae. The handle 115 is then actuated, advancing the VBR implant 140 distally, distracting the adjacent vertebrae 175, as shown in FIG. 7B. In some embodiments, the VBR implant 140 is fully inserted into disk space after depressing the handle twelve (12) times. The paddle tips 125 of the instrument are automatically removed from the vertebral bodies as the VBR implant 140 is inserted. This is performed by way of end posts 160 on the inserter shaft that contact the vertebrae, and with continued advancement, push out the instrument disk space, as shown in FIG. 7C. This may be done during the final three depressions of the action lever 115. Final implant release occurs upon turning the back knob 150 in a counter clockwise manner, unscrewing the inserter shaft 110 from the VBR implant 140.

Example embodiments of the methods and components of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments, but should be defined only in accordance with the following claims and their equivalents.

What is claimed:

1. An instrument for use in an anterior lumbar interbody fusion (ALIF) procedure for distraction of adjacent vertebrae and insertion of a vertebral body replacement (VBR) implant, the instrument comprising:
a hollow body having a proximal end and a distal end;
an inserter shaft positioned within the body configured to removably engage a VBR implant near the distal end of the body;
an actuatable handle coupled to the body, the handle being configured to engage the inserter shaft to advance the VBR implant in a distal direction during handle actuation; and
a pair of opposed distraction arms removably coupled to the distal end of the body, the distraction arms having paddle tips configured to fit between adjacent vertebrae, the distraction arms being movable from a closed position to an open position during distal advancement of the VBR implant between the distraction arms.

2. The instrument of claim 1, wherein the opposed distraction arms include opposed ramps configured to contact the VBR implant during distal advancement to move the opposed distraction arms from the closed position to the open position.

3. The instrument of claim 1, wherein the paddle tips are selected to match a width of the desired VBR implant.

4. The instrument of claim 1, wherein each distraction arm includes a depressible button configured for coupling/uncoupling the distraction arm from the body.

5. The instrument of claim 1, wherein the distraction arms are made of titanium or aluminum.

6. The instrument of claim 1, wherein the inserter shaft further includes endposts proximate the VBR implant, the endposts being configured to contact the adjacent vertebrae once the VBR implant is positioned between the adjacent vertebrae.

7. The instrument of claim 1, wherein the handle actuation includes depressing the handle toward the body, each handle depression advancing the VBR implant distally at least 1 cm.

8. The instrument of claim 1, further comprising a handle return spring coupled to the handle and body, the return spring being configured to return the handle to a start position after each actuation.

9. The instrument of claim 1, further comprising a knob coupled to the inserter shaft near the proximal end of the body, the knob being configured to rotate the shaft to engage or disengage the VBR implant.

10. The instrument of claim 1, wherein the inserter shaft includes threads configured for a threaded engagement with the VBR implant.

11. A system for use in an anterior lumbar interbody fusion (ALIF) procedure for distraction of adjacent vertebrae and insertion of a vertebral body replacement (VBR) implant, the system comprising:
a hollow body having a proximal end and a distal end;
an inserter shaft positioned within the body, the inserter shaft having threads on a distal end;
a VBR implant in threaded engagement with inserter shaft;
a knob coupled to the inserter shaft near the proximal end of the body, the knob being configured to rotate the shaft to engage or disengage the VBR implant;
an actuatable handle coupled to the body, the handle being configured to engage the inserter shaft to advance the VBR implant in a distal direction during handle actuation; and
a pair of opposed distraction arms removably coupled to the distal end of the body, the distraction arms having:
paddle tips configured to fit between adjacent vertebrae;
opposed ramps configured to contact the VBR implant during distal advancement to move the opposed distraction arms from a closed position to an open position; and;
depressible buttons configured for coupling/uncoupling the distraction arms from the body.

12. The system of claim 11, wherein the paddle tips are selected to match a width of the VBR implant.

13. The system of claim 11, wherein the distraction arms are made of titanium or aluminum.

14. The system of claim 11, wherein the inserter shaft further includes endposts proximate the VBR implant, the endposts being configured to contact the adjacent vertebrae once the VBR implant is positioned between the adjacent vertebrae.

15. The system of claim 11, wherein the handle actuation includes depressing the handle toward the body, each handle depression advancing the VBR implant distally at least 1 cm.

16. The system of claim 11, further comprising a handle return spring coupled to the handle and body, the return spring being configure to return the handle to a start position after each actuation.

17. A method of distracting adjacent vertebrae and inserting a vertebral body replacement (VBR) implant between adjacent vertebrae during an anterior lumbar interbody fusion (ALIF) procedure, the method comprising:
positioning a VBR implant in an instrument, the instrument comprising:
a hollow body having a proximal end and a distal end;
an inserter shaft positioned within the body configured to removably engage the VBR implant;
an actuatable handle coupled to the body, the handle being configured to engage the inserter shaft to advance the VBR implant in a distal direction during handle actuation; and
a pair of opposed distraction arms removably coupled to the distal end of the body, the distraction arms having paddle tips configured to fit between adjacent vertebrae, the distraction arms being movable from a closed position to an open position during distal advancement the VBR implant between the distraction arms;
placing the paddle tips of the distraction arms between the adjacent vertebrae;
distracting the adjacent vertebrae with the paddle tips as the distraction arms move from the closed position to the open position;
inserting the VBR implant between the adjacent vertebrae;
disengaging the VBR implant from inserter shaft; and
removing the paddle tips of the inserter/disctractor instrument from the adjacent vertebrae.

18. The method of claim 17, wherein the opposed distraction arms include opposed ramps configured to contact the VBR implant during distal advancement, wherein moving the distraction arms from the closed position to the open position includes actuating the handle and advancing the VBR implant between the distraction arms in contact with the ramps.

19. The method of claim 17, wherein disengaging the VBR implant from inserter shaft includes rotating the inserter shaft with a knob to disengage a threaded engagement with the VBR implant.

20. The method of claim 17, further comprising selecting distraction arms having paddle tips sized to match a width of the desired VBR implant and coupling the distraction arms to the body.
